(19) United States
(12) Patent Application Publication
(10) Pub. No.: US 2023/0268049 A1
(43) Pub. Date: Aug. 24, 2023

(54) PHARMACY PLATFORM SYSTEM FOR PROVIDING ELECTROCEUTICAL SERVICE BASED ON ONLINE AND OFFLINE AND METHOD THEREOF

(71) Applicant: Precintel., Inc., Seoul (KR)

(72) Inventors: Dong-Joo KIM, Seoul (KR); Young-Tak KIM, Goyang-si (KR)

(21) Appl. No.: 18/106,471

(22) Filed: Feb. 6, 2023

(30) Foreign Application Priority Data
Feb. 23, 2022 (KR) 10-2022-0023791
Dec. 20, 2022 (KR) 10-2022-0179488

(51) Int. Cl. G16H 20/30; G16H 40/20; G06F 40/268; G06F 40/35; G06F 40/295

(52) U.S. Cl. CPC G16H 20/30; G16H 40/20; G06F 40/268; G06F 40/35; G06F 40/295

(57) ABSTRACT

Provided is a pharmacy platform system for providing an online and offline based electroceutical service and a method thereof. The method for providing an electroceutical service using a system for providing an online and offline based electroceutical service includes receiving, by an electroceutical pharmacy server, an input electroceutical prescription from a user terminal possessed by a user who accesses an electroceutical pharmacy site or visits the electroceutical pharmacy; checking a stock status of an electroceutical included in the prescription; transmitting a possible supply date of the electroceutical to the user terminal according to the identified stock status, and requesting a pickup information input; and providing the corresponding electroceutical according to input pickup information when a purchase cost of the corresponding electroceutical is paid and the pickup information including a pickup date and a pickup method is input from the user terminal.

### CROSS-REFERENCE TO RELATED APPLICATIONS

The present application claims priority under 35 USC 119 to Korean patent application Nos. 10-2022-0023791 filed on Feb. 23, 2022, and 10-2022-0179488 filed on Dec. 20, 2022, the entire contents of which are incorporated herein by reference.

### BACKGROUND

### 1. Field

The present disclosure relates to a pharmacy platform system for providing an online and offline based electroceutical service and a method thereof, and more particularly, to a pharmacy platform system for providing an online and offline based electroceutical service to provide a prescription electroceutical from a doctor to a user who visits an electroceutical pharmacy offline or accesses the electroceutical pharmacy online and a method thereof.

### 2. Description of the Related Art

Electroceuticals have become a hot topic in research in recent years. The term “electroceutical” results from combining “electronic” with “pharmaceutical”, and the concept of electroceuticals is the treatment of diseases through safe electrical stimulation instead of medication or injection.

Electroceuticals are a medical device that applies microcurrent stimulation to the locations of diseases or the neural networks, and provide medical treatments or evidence based treatments, making it completely distinct from other common wellness devices.

In the 2018 world economic forum annual meeting, electroceuticals were considered as one of the top ten emerging technologies and many people acknowledge the potential growth and possibility of electroceuticals. The reason that electroceuticals are attracting attention is the risk of side effects of oral and injectable medications and high costs of new medications that pose a big financial burden on patients. Additionally, the existing oral medications are susceptible to wrong time administration errors or side effects on unintended parts of the body, while electroceuticals stimulate only specific nerves in a safe manner, thereby significantly reducing the risk of side effects. Further, electroceuticals can be effectively used for the treatment of chronic diseases and incurable and untreatable diseases such as Parkinson's disease, so many startups are entering the market. Accordingly, the electroceutical market is also growing fast all over the world.

Electroceuticals are gaining attention as the next-generation medicine that will change the concept of medicine in the future due to less side effects and lower development costs than the existing medicine. However, there is a concern for the electroceutical technology, as along with high expectations. Above all, what is required to introduce the electroceutical technology into practical applications at the present time is a system for allowing physicians to appropriately prescribe and administer electroceuticals to patients, but such platform systems have never been suggested so far.

Besides, despite many expected advantages of electroceuticals in the clinical applications, additional duties of doctors such as in-depth knowledge of patients and burden of teaching patients on method of use, and conservative views on the electroceutical technology may reduce the prescription rate of electroceuticals and limit the practical use.

Accordingly, there is a need for the development of technology to provide an online and offline based electroceutical pharmacy service for providing patients with prescription electroceuticals and usage guidelines together and achieving continuous distribution through electroceutical manufacturers.

### SUMMARY

The present disclosure is directed to providing a pharmacy platform system for providing an online and offline based electroceutical service to provide a prescription electroceutical from a doctor to a user who visits an electroceutical pharmacy offline or accesses the electroceutical pharmacy online and a method thereof.

The present disclosure is further directed to providing a pharmacy platform system for providing an online and offline based electroceutical service to provide services that check an electroceutical stock status in real time, receive an out of stock electroceutical from an electroceutical manufacturer, and when an electroceutical is out of order, request an after-sales service to the corresponding electroceutical manufacturer and send the repaired electroceutical to the corresponding user and a method thereof.

To achieve the above-described technical objective, a method for providing an electroceutical service using a pharmacy platform system for providing an online and offline based electroceutical service according to an embodiment of the present disclosure includes receiving, by an electroceutical pharmacy server, an input electroceutical prescription from a user terminal possessed by a user who accesses an electroceutical pharmacy site or visits the electroceutical pharmacy; checking a stock status of an electroceutical included in the prescription; transmitting a possible supply date of the electroceutical to the user terminal according to the identified stock status, and requesting a pickup information input; and providing the corresponding electroceutical according to input pickup information when a purchase cost of the electroceutical is paid and the pickup information including a pickup date and a pickup method is input from the user terminal.

In this instance, checking the stock status may include requesting electroceutical supply and an expected supply date to an electroceutical manufacturer server of the corresponding electroceutical when the electroceutical is out of stock.

Additionally, requesting the pickup information input may include providing the user terminal with the possible supply date of the electroceutical in a calendar or list view to request the pickup information input, and providing the electroceutical may include booking parcel delivery dispatch to a pre-stored address included in a user information database to be delivered on the input pickup date when the input pickup method is parcel delivery, and providing the user terminal with store information including an address and contact number of the electroceutical pharmacy store when the input pickup method is the user's visit and pickup.

Additionally, providing the electroceutical may include providing usage guidelines of the corresponding electroceutical together according to an input device description method from the user terminal when providing the electroceutical.

Additionally, the method for providing an electroceutical service may further include receiving, by the electroceutical pharmacy server, an input after-sales service request of the electroceutical from the user terminal; determining whether an after-sales service is possible or not by referring to a purchase date and electroceutical information of the corresponding electroceutical included in purchase history information of a user information database; when the after-sales service is possible as a result of the determination, requesting the after-sales service of the corresponding electroceutical to an electroceutical manufacturer server of the corresponding electroceutical, and receiving after-sales service completion information including an expected after-sales service completion date from the electroceutical manufacturer server; and providing the user terminal with the received after-sales service completion information, and requesting the pickup information input to the user terminal.

Additionally, checking the stock status may include, when the electroceutical is out of stock, generating a recommendation list of electroceuticals currently available for supply in an order of similarity of performance and efficacy information to the electroceutical by analyzing the performance and efficacy information of the corresponding electroceutical and referring to a stock status database, and providing the generated recommendation list to the user terminal.

Additionally, a pharmacy platform system for providing an online and offline based electroceutical service according to another embodiment of the present disclosure includes a user terminal possessed by a user who accesses an electroceutical pharmacy site or visits the electroceutical pharmacy to input a prescription from a doctor or an after-sales service request of an electroceutical that the user has purchased; an electroceutical pharmacy server to transmit a possible supply date of the electroceutical to the user terminal according to a stock status of the electroceutical included in the prescription, request an pickup information input and provide the electroceutical according to input pickup information; and an electroceutical manufacturer server to provide electroceutical supply information to the electroceutical pharmacy server, and respond to the after-sales service request of the electroceutical.

In this instance, the electroceutical pharmacy server may include an input unit to receive the input electroceutical prescription from the user terminal; a stock status checking unit to check the stock status of the electroceutical included in the prescription by referring to a stock status database; an information providing and request unit to transmit the possible supply date of the electroceutical to the user terminal according to the identified stock status, and request the pickup information input; and an electroceutical providing unit to provide the electroceutical according to the input pickup information when the pickup information including a pickup date and a pickup method is input from the user terminal and a purchase cost of the electroceutical is paid.

Additionally, the information providing and request unit may provide the user terminal with the possible supply date of the electroceutical in a calendar or list view to request the pickup information input, and the electroceutical providing unit may book parcel delivery dispatch to a pre-stored address included in a user information database to be delivered on the input pickup date when the input pickup method is parcel delivery, and may provide the user terminal with store information including an address and contact number of the electroceutical pharmacy store when the input pickup method is the user's visit and pickup.

Additionally, the stock status checking unit may request electroceutical supply and an expected supply date to an electroceutical manufacturer server of the corresponding electroceutical when the electroceutical is out of stock, and the electroceutical providing unit may provide usage guidelines of the corresponding electroceutical together according to an input device description method from the user terminal when providing the electroceutical.

Additionally, the electroceutical pharmacy server may further include an after-sales service unit to determine whether an after-sales service is possible or not by referring to a purchase date and electroceutical information of the corresponding electroceutical included in purchase history information of a user information database when an electroceutical after-sales service request is received from the user terminal, and when the after-sales service is possible, to request the after-sales service of the corresponding electroceutical to an electroceutical manufacturer server of the corresponding electroceutical, receive after-sales service completion information including an expected after-sales service completion date from the electroceutical manufacturer server and provide the after-sales service completion information to the user terminal, and request the pickup information input to the user terminal.

Additionally, when the electroceutical is out of stock, the stock status checking unit may generate a recommendation list of electroceuticals currently available for supply in an order of similarity of performance and efficacy information to the corresponding electroceutical by analyzing the performance and efficacy information of the electroceutical and referring to the stock status database, and provide the generated recommendation list to the user terminal.

According to the present disclosure, it is possible to provide a prescription electroceutical from a doctor to a user who visits the electroceutical pharmacy offline or accesses the electroceutical pharmacy online together with the detailed usage guidelines of the electroceutical, thereby reducing the physician's duties imposed in the introduction of electroceuticals, and contributing to the increased prescription rate of electroceuticals.

Additionally, according to the present disclosure, it is possible to provide the service that checks an electroceutical stock status in real time, is supplied with an out of stock electroceutical from an electroceutical manufacturer, and when an electroceutical is out of order, requests an after-sales service to the corresponding electroceutical manufacturer and sends the repaired electroceutical to the corresponding user, thereby forming a continuous electroceutical distribution and supply chain of the electroceutical manufacturer and providing the user's convenience.

Furthermore, according to the present disclosure, it is possible to satisfy the needs of the doctor, the electroceutical manufacturer and the user, by acting as connecting links among the doctor, the electroceutical manufacturer and the user with respect to the electroceutical pharmacy running online and offline.

### BRIEF DESCRIPTION OF THE DRAWINGS

FIG. 1 is an architecture diagram of a pharmacy platform system for providing an online and offline based electroceutical service according to an embodiment of the present disclosure.

FIG. 2 is a block diagram showing an electroceutical pharmacy server of FIG. 1.

FIG. 3 is a flowchart showing the sequence of steps of a method for providing an online and offline based electroceutical service according to an embodiment of the present disclosure.

FIG. 4 is a schematic diagram illustrating a method for providing an online and offline based electroceutical service according to an embodiment of the present disclosure.

### DETAILED DESCRIPTION

Hereinafter, exemplary embodiments of the present disclosure will be described in more detail with reference to the accompanying drawings. In this instance, the thickness of lines or the size of elements shown in the drawings may be exaggerated for clarity and convenience of description.

Additionally, the terms as used herein are defined in the light of functions in the present disclosure, but may vary depending on the intention of users or operators or the convention. Accordingly, the definition of these terms should be made based on the context throughout the specification.

The term “unit” or “module” are defined herein as having its broadest definition to ordinary skill in the art, including software including instructions executable in a non-transitory computer-readable medium that would perform the associated function when executed, a circuit designed to perform the associated function, hardware designed to perform the associated function, or a combination of software, a circuit, or hardware designed to perform the associated function.

Further, it is to be understood that all detailed descriptions mentioning specific embodiments of the present disclosure as well as principles, aspects, and embodiments of the present disclosure are intended to include structural and functional equivalences thereof. Further, it is to be understood that these equivalences include an equivalence that will be developed in the future as well as an equivalence that is currently well-known, that is, all elements invented so as to perform the same function regardless of a structure.

Therefore, it is to be understood that, for example, block diagrams of the present specification illustrate a conceptual aspect of an illustrative circuit for embodying a principle of the present disclosure. Therefore, it is to be understood that all flow charts, state transition diagrams, pseudo-codes, and the like, illustrate various processes that may be tangibly embodied in a computer-readable medium and that are executed by computers or processors regardless of whether or not the computers or the processors are clearly illustrated.

Functions of various elements including processors or functional blocks represented as concepts similar to the processors and illustrated in the accompanying drawings may be provided using hardware having capability to execute software in connection with appropriate software as well as dedicated hardware. When the functions are provided by the processors, they may be provided by a single dedicated processor, a single shared processor, or a plurality of individual processors, and some of them may be shared with each other.

In addition, the explicit use of terms presented as the processor, control, or similar concepts should not be interpreted exclusively by quoting hardware capable of executing software, but should be understood to implicitly include, without limitation, digital signal processor (DSP) hardware, a ROM for storing software, a RAM, and a non-volatile memory. The above-mentioned terms may also include well-known other hardware.

In the claims of the present specification, components represented as means for performing functions mentioned in a detailed description are intended to include all methods for performing functions including all types of software including, for example, a combination of circuit devices performing these functions, firmware/micro codes, or the like, and are coupled to appropriate circuits for executing the software so as to execute these functions. It is to be understood that since functions provided by variously mentioned means are combined with each other and are combined with a method demanded by the claims in the present disclosure defined by the claims, any means capable of providing these functions are equivalent to means recognized from the present specification.

Hereinafter, exemplary embodiments of the present disclosure will be described in more detail with reference to the accompanying drawings.

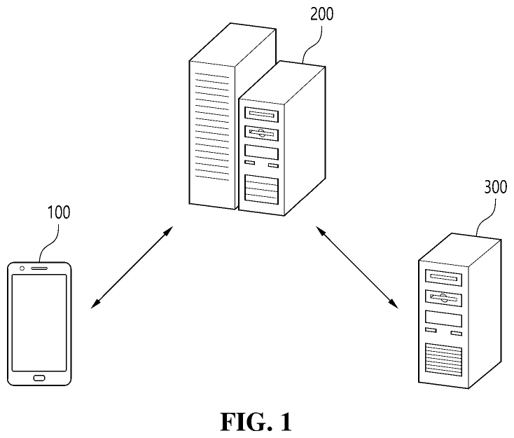

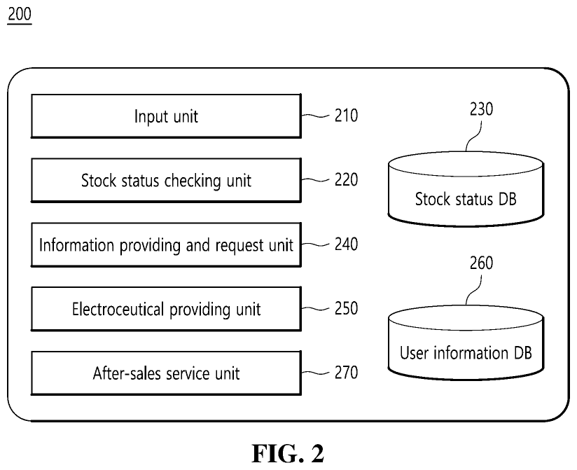

To begin with, a pharmacy platform system for providing an online and offline based electroceutical service according to an embodiment of the present disclosure will be described through FIGS. 1 and 2.

FIG. 1 is a system architecture diagram showing the pharmacy platform system for providing an online and offline based electroceutical service according to an embodiment of the present disclosure.

As shown in FIG. 1, the pharmacy platform system for providing an online and offline based electroceutical service according to an embodiment of the present disclosure includes a user terminal 100, an electroceutical pharmacy server 200 and an electroceutical manufacturer server 300.

First, the user terminal 100 is possessed by a user who accesses an electroceutical pharmacy site or visits an electroceutical pharmacy and is used to input a prescription from a doctor or an after-sales service request of an electroceutical that the user has purchased.

In this instance, in the case of an online service, the user terminal 100 may be a smartphone, a PC, a tablet and a notebook computer capable of making access to the electroceutical pharmacy site, and in the case of an offline service, the user terminal 100 may be an input-only terminal equipped in the electroceutical pharmacy store.

Additionally, electroceuticals mentioned in an embodiment of the present disclosure are a word made by blending electronic and pharmaceutical and refer to an instrument or device for the treatment of diseases through safe electrical stimulation instead of medication or injection.

The electroceutical pharmacy server 200 transmits the possible supply date of the electroceutical to the user terminal 100 according to the stock status of the electroceutical included in the input prescription received through the user terminal 100, and when the purchase cost of the corresponding electroceutical is paid from the user terminal 100, requests a pickup information input to the user terminal 100 and provides the electroceutical according to the input pickup information.

In this instance, the pickup information essentially includes a pickup date and a pickup method, and to make the user easier to input, a selection input box may be provided to allow the user to select and input the dates on which the user can pick up the corresponding electroceutical and whether the user wants the electroceutical to be delivered or will pick up the electroceutical through the user terminal 100.

The electroceutical pharmacy server is a server provided in the electroceutical pharmacy, and may receive an electroceutical related clinical evidence inquiry from the pharmacist or the doctor, provide query-based related information from a connected external server, or collect input after-sales service information processing information from the pharmacist on a preset after-sales service information collection DB and provide after-sales service information reporting.

The electroceutical manufacturer server 300 provides electroceutical supply information to the electroceutical pharmacy server 200, and responds to the after-sales service request of the electroceutical.

In this instance, the electroceutical manufacturer server 300 is a server of an electroceutical distributor and supplier company to the electroceutical pharmacy, and supplies the electroceutical or provides the after-sales service of the electroceutical in response to the request received from the electroceutical pharmacy server 200.

Hereinafter, the electroceutical pharmacy server 200 according to an embodiment of the present disclosure will be described in detail.

FIG. 2 is a block diagram showing the electroceutical pharmacy server of FIG. 1.

As shown in FIG. 2, the electroceutical pharmacy server 200 according to an embodiment of the present disclosure includes an input unit 210, a stock status checking unit 220, an information providing and request unit 240, an electroceutical providing unit 250 and an after-sales service unit 270.

First, the input unit 210 receives the input electroceutical prescription from the user terminal 100.

Additionally, the stock status checking unit 220 checks the stock status of the electroceutical included in the input prescription from the user terminal 100 by referring to a stock status database 230.

In this instance, when the corresponding electroceutical is out of stock, the stock status checking unit 220 may request electroceutical supply and the expected supply date to the electroceutical manufacturer server 300 of the corresponding electroceutical.

Accordingly, the electroceutical manufacturer server 300 may transmit the possible supply date of the electroceutical requested from the electroceutical pharmacy server 200 to the electroceutical pharmacy server 200.

Additionally, when the corresponding electroceutical is out of stock, the stock status checking unit 220 may generate a recommendation list of electroceuticals currently available for supply in an order of similarity of performance and efficacy information to the corresponding electroceutical by analyzing the performance and efficacy information of the electroceutical and referring to the stock status database 230, and provide the generated recommendation list to the user terminal 100.

In this instance, the stock status checking unit 220 may provide the recommendation list displaying the degree of performance and efficacy similarity to the user's desired electroceutical as score or percentage for each of the electroceuticals included in the recommendation list.

Accordingly, when any one of the similar electroceuticals is selected from the user terminal 100 having received the recommendation list, the corresponding electroceutical may be provided to the user.

That is, according to an embodiment of the present disclosure, when the prescription electroceutical from the doctor is out of stock, it is possible to provide the user with the list of similar electroceuticals to the corresponding electroceutical, thereby providing the user with the electroceutical quickly when the user has an urgent need for the electroceutical.

Additionally, the information providing and request unit 240 transmits the possible supply date of the electroceutical to the user terminal 100 according to the stock status identified by the stock status checking unit 220, and requests the pickup information input.

In this instance, the information providing and request unit 240 may provide the user terminal 100 with the possible supply date of the electroceutical in a calendar or list view to request the pickup information input.

Accordingly, it is possible to allow the user to select and input a desired date from the possible dates on which the electroceutical will be provided, and select a method for receiving the electroceutical.

The information providing and request unit 240 may provide electroceutical-related clinical information.

When the information providing and request unit 240 receives an inquiry about the information associated with electroceutical-related clinical evidence and efficacy, the information providing and request unit 240 may search for the corresponding information from the connected external server and transmit appropriate information. In this instance, the connected external server may include, for example, an online academic DB in which disease names, electroceutical names, guidelines, and efficacy information are stored, but is not limited thereto.

To this end, the information providing and request unit 240 may include a preprocessing module, a query analysis module, and an information search module.

The preprocessing module may perform natural language preprocessing on the text of the inquiry from a doctor and a pharmacist. In this instance, the natural language preprocessing may include morphological analysis, and roots extraction and analysis.

The query analysis module is configured to perform semantic analysis of the preprocessed text, and may include a transformer-based named entity recognition model. The transformer-based named entity recognition model may include a neural network that learns context and meaning of a sentence by tracking relationships in sequential data such as words in the sentence, and may detect even subtle changes in meaning by analyzing the meaning and relationship of distant elements in the text using the self-attention technique.

The information search module may process the user's desired response in real time according to a preset information search algorithm. More specifically, the information search module may perform keyword matching in the external server based on the named entity recognized by the query analysis module, and make a response to the inquiry based on the matched keyword. In this instance, the information search module may make a response to the inquiry in a natural dialogue format.

Additionally, the electroceutical providing unit 250 calculates the purchase cost of the corresponding electroceutical, and when the pickup information including the pickup date and the pickup method is input from the user terminal 100 and the purchase cost of the corresponding electroceutical is paid, provides the corresponding electroceutical according to the input pickup information.

In this instance, when the input pickup method from the user terminal 100 is parcel delivery, the electroceutical providing unit 250 books parcel delivery dispatch to a pre-stored address included in a user information database 260 to be delivered on the input pickup date, and when the input pickup method is the user's visit and pickup, provides the user terminal 100 with store information including address and contact number of the corresponding electroceutical pharmacy store.

Additionally, when providing the electroceutical, the electroceutical providing unit 250 may provide the usage guidelines of the corresponding electroceutical together according to the input device description method from the user terminal 100.

For example, when the online device description is input from the user terminal 100, the user terminal 100 may be allowed to select and input chatting and telephone consultation and the usage guidelines of the corresponding electroceutical may be provided according to the input method.

Finally, when the after-sales service request of the electroceutical is input from the user terminal 100, the after-sales service unit 270 may determine whether the after-sales service is possible or not by referring to the purchase date and electroceutical information of the corresponding electroceutical included in purchase history information of the user information database 260, and when the after-sales service is possible, requests the after-sales service of the corresponding electroceutical to the electroceutical manufacturer server 300 of the corresponding electroceutical.

In this instance, the after-sales service unit 270 may determine whether the warranty period has passed or not by referring to the electroceutical information of the corresponding electroceutical, and when the warranty period has passed, may output a message “The warranty period of the corresponding electroceutical has passed, and the repair cost may be charged.” to the user terminal 100.

Additionally, the after-sales service unit 270 receives after-sales service completion information including the expected after-sales service completion date from the electroceutical manufacturer server 300 and provides the after-sales service completion information to the corresponding user terminal 100, and requests the pickup information input.

In this instance, the electroceutical manufacturer server 300 having received the after-sales service request calculates the expected after-sales service completion date of the corresponding electroceutical and transmits the same to the electroceutical pharmacy server 200, and the after-sales service unit 270 having received the expected after-sales service completion date provides the user terminal 100 having requested the after-sales service with the after-sales service completion information including the expected after-sales service completion date and the repair cost and requests the pickup information input including the pickup date and the pickup method.

The after-sales service unit 270 may continuously store the input after-sales service information from the pharmacist in an after-sales service information collection DB.

The after-sales service unit 270 may be connected to the after-sales service information collection DB in which the after-sales service information is collected, to periodically transmit reports for each after-sales service type to the electroceutical manufacturer server 300. Through this, the electroceutical manufacturer may conduct a more systematic after-sales service analysis.

More specifically, the after-sales service unit 270 may cluster after-sales services by type through a preset clustering algorithm and transmit reports for each classified after-sales service type to the electroceutical manufacturer server 300. In this instance, the preset clustering algorithm may be density-based spatial clustering of applications with noise (DBSCAN), and the clustering algorithm may cluster after-sales services by type by converting a list of after-sales service main issues to vector using Word2Vec and mapping on a 2-dimensional space.

In this instance, when the pickup information including the pickup date and the pickup method is input from the user terminal 100, the electroceutical providing unit 250 provides the corresponding electroceutical according to the input pickup information.

That is, in the same way as providing a newly purchased electroceutical, when the input pickup method from the user terminal 100 is parcel delivery, the electroceutical providing unit 250 books parcel delivery dispatch to the pre-stored address included in the user information database 260 to be delivered on the input pickup date, and when the input pickup method is the user's visit and pickup, provides the user terminal 100 with store information including address and contact number of the corresponding electroceutical pharmacy store.

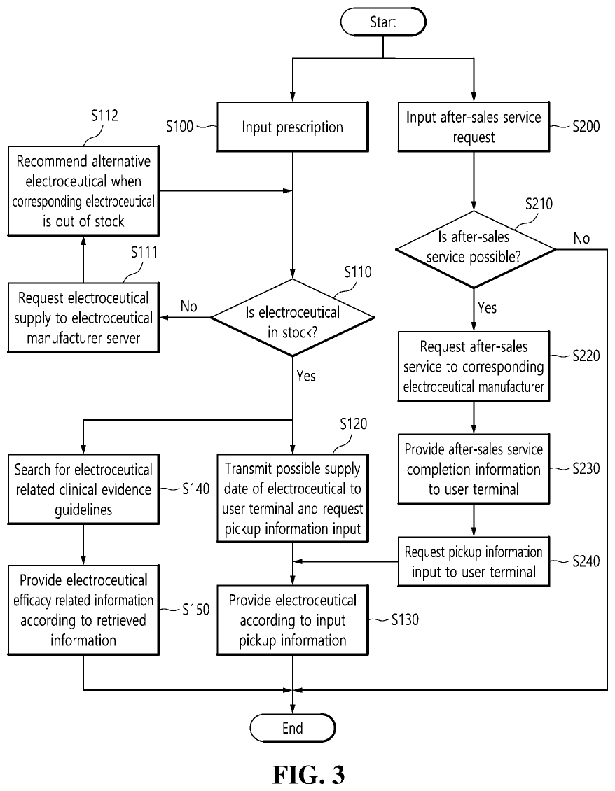

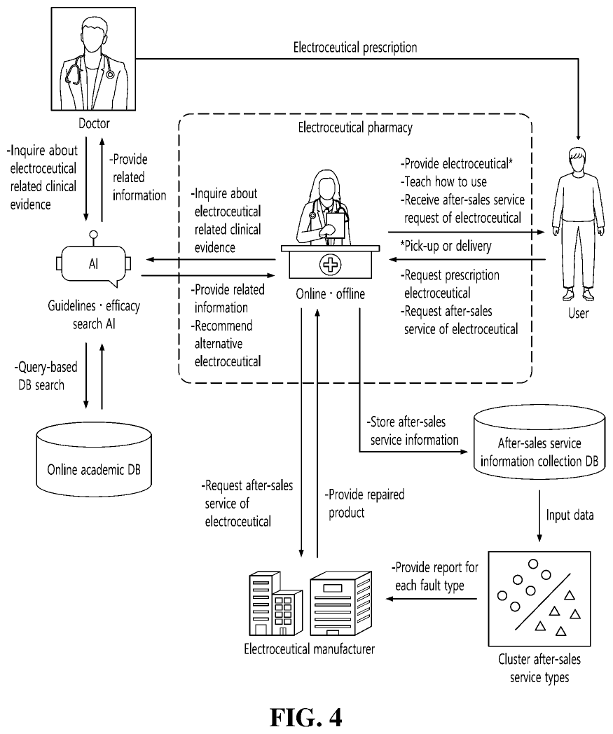

Hereinafter, a method for providing an online and offline based electroceutical service according to an embodiment of the present disclosure will be described through FIGS. 3 and 4.

FIG. 3 is a flowchart showing the sequence of steps of the method for providing an online and offline based electroceutical service according to an embodiment of the present disclosure, and the operation of the present disclosure will be described in detail with reference to FIG. 3.

According to an embodiment of the present disclosure, first, the input unit 210 of the electroceutical pharmacy server 200 receives the input electroceutical prescription from the user terminal 100 possessed by the user who accesses the electroceutical pharmacy site or visits the electroceutical pharmacy (S100).

In this instance, in the case of an online service, the user terminal 100 may be a smartphone, a PC, a tablet and a notebook computer capable of making access to the electroceutical pharmacy site, and in the case of an offline service, the user terminal 100 may be an input-only terminal equipped in the electroceutical pharmacy store.

Subsequently, the stock status checking unit 220 checks the stock status of the electroceutical included in the input prescription received in the step S100 (S110).

As a result of determination in the step S110, when the corresponding electroceutical is out of stock, the stock status checking unit 220 requests the electroceutical supply and the expected supply date to the electroceutical manufacturer server 300 of the corresponding electroceutical (S111).

Additionally, as a result of determination in the step S110, when the corresponding electroceutical is out of stock, the stock status checking unit 220 may generate the recommendation list of electroceuticals currently available for supply in an order of similarity of performance and efficacy information to the corresponding electroceutical by analyzing the performance and efficacy information of the electroceutical and referring to the stock status database 230, and provide the generated recommendation list to the user terminal 100 (S112).

In this instance, the stock status checking unit 220 may provide the recommendation list displaying the degree of performance and efficacy similarity to the user's desired electroceutical as score or percentage for each of the electroceuticals included in the recommendation list.

Additionally, when the corresponding electroceutical is in stock, the information providing and request unit 240 transmits the possible supply date of the electroceutical to the user terminal 100 and requests the pickup information input (S120).

In the step S120, the information providing and request unit 240 may provide the user terminal 100 with the possible supply date of the electroceutical in a calendar or list view to request the pickup information input.

When the pickup information including the pickup date and the pickup method from the user terminal 100 is input in response to the request in the step S120, the electroceutical providing unit 250 provides the corresponding electroceutical according to the input pickup information (S130).

In this instance, when the input pickup method from the user terminal 100 is parcel delivery, the electroceutical providing unit 250 may book parcel delivery dispatch to the pre-stored address included in the user information database 260 to be delivered on the input pickup date, and when the input pickup method is the user's visit and pickup, may provide the user terminal 100 with the store information including the address and contact number of the corresponding electroceutical pharmacy store.

Additionally, when providing the electroceutical, the electroceutical providing unit 250 may provide the usage guidelines of the corresponding electroceutical together according to the input device description method from the user terminal 100. To this end, the electroceutical providing unit 250 may search for electroceutical related clinical evidence guidelines (S140), and provide electroceutical efficacy related information according to the retrieved information (S150).

Additionally, when the input unit 210 of the electroceutical pharmacy server 200 receives the after-sales service request of the electroceutical from the user terminal 100 possessed by the user who accesses the electroceutical pharmacy site or visits the electroceutical pharmacy (S200), the after-sales service unit 270 determines whether the after-sales service is possible or not by referring to the purchase date and the electroceutical information of the corresponding electroceutical included in the purchase history information of the user information database 260 (S210).

As a result of determination in the step S210, when the after-sales service is possible, the after-sales service unit 270 requests the after-sales service of the corresponding electroceutical to the electroceutical manufacturer server 300 of the corresponding electroceutical (S220).

Additionally, the after-sales service unit 270 receives the after-sales service completion information including the expected after-sales service completion date from the corresponding electroceutical manufacturer server 300 and provides the received after-sales service completion information to the user terminal 100 having requested the after-sales service (S230).

Additionally, the after-sales service unit 270 requests the pickup information input to the user terminal 100 (S240).

When the pickup information including the pickup date and the pickup method is input from the user terminal 100 in response to the request in the step S240, the electroceutical providing unit 250 provides the corresponding electroceutical according to the input pickup information.

That is, in the same way as providing a newly purchased electroceutical, when the input pickup method from the user terminal 100 is parcel delivery, the electroceutical providing unit 250 books parcel delivery dispatch to the pre-stored address included in the user information database 260 to be delivered on the input pickup date, and when the input pickup method is the user's visit and pickup, provides the user terminal 100 with the store information including the address and contact number of the corresponding electroceutical pharmacy store.

FIG. 4 is a schematic diagram illustrating the method for providing an online and offline based electroceutical service according to an embodiment of the present disclosure.

Describing the method for providing an online and offline based electroceutical service according to an embodiment of the present disclosure with reference to FIG. 4, first, the doctor gives the electroceutical prescription to the user in the same way as the common medical routine.

Additionally, the user who gets the electroceutical prescription from the doctor visits the electroceutical pharmacy store in person or accesses the electroceutical pharmacy site using his/her user terminal 100.

That is, the user may purchase the necessary electroceutical from the online or offline electroceutical pharmacy.

Accordingly, the user may be provided with the necessary electroceutical through the electroceutical pharmacy store or the electroceutical pharmacy site, and may request the after-sales service when the electroceutical in use is out of order.

Additionally, when the user accesses the electroceutical pharmacy site provided by the electroceutical pharmacy server 200 online through the user terminal 100 or visits the offline electroceutical pharmacy in person, the electroceutical pharmacy guides the user to input the prescription through the user terminal 100 or the dedicated terminal equipped in the electroceutical pharmacy store, and provides the corresponding electroceutical.

Additionally, the electroceutical pharmacy performs the role in providing the electroceutical and detailed usage guidelines to the user through the user terminal 100, receiving the after-sales service request of the electroceutical from the user and transmitting the request to the corresponding electroceutical manufacturer server 300, and selling the electroceutical supplied from the electroceutical manufacturer via online delivery or in the offline store.

Additionally, when the electroceutical pharmacy receives inquiries about electroceutical related clinical evidence from the doctor and the pharmacist, the electroceutical pharmacy may provide related information retrieved through an AI-based search engine to the doctor and the pharmacist.

Additionally, when the necessary electroceutical is out of stock, the electroceutical pharmacy may recommend an alternative electroceutical.

Additionally, the electroceutical pharmacy may store electroceutical after-sales service information in the after-sales service information collection DB, cluster after-sales service types using the same and provide reports for each after-sales service type to the electroceutical manufacturer.

Finally, the electroceutical manufacturer manages the electroceutical through the electroceutical manufacturer server 300, continuously distributes and supplies the electroceutical to the corresponding electroceutical pharmacy by the communication with the electroceutical pharmacy server 200, and provides the after-sales service of the electroceutical device in response to the received request.

The method for providing an online and offline based electroceutical service may be implemented as an application or in the format of program instructions that may be executed through a variety of computer components and may be recorded in computer readable recording media. The computer readable recording media may include program instructions, data files and data structures alone or in combination.

The program instructions recorded in the computer readable recording media are specially designed and configured for the present disclosure and may be program instructions known and available to those having ordinary skill in the field of computer software.

Examples of the computer readable recording media include hardware devices specially designed to store and execute the program instructions, for example, magnetic media such as hard disk, floppy disk and magnetic tape, optical media such as CD-ROM and DVD, magneto-optical media such as floptical disk, and ROM, RAM and flash memory.

Examples of the program instructions include machine code generated by a compiler as well as high-level language code that can be executed by a computer using an interpreter. The hardware device may be configured to act as one or more software modules to perform the processing according to the present disclosure.

As described above, the method for providing an online and offline based electroceutical service according to an embodiment of the present disclosure provides the prescription electroceutical from the doctor to the user who visits the electroceutical pharmacy offline or accesses the electroceutical pharmacy online together with the detailed usage guidelines of the electroceutical, thereby reducing the physician's duties imposed in the introduction of electroceuticals, and contributing to the increased prescription rate of electroceuticals.

Additionally, according to an embodiment of the present disclosure, it is possible to provide the services that check the electroceutical stock status in real time, receive the out of stock electroceutical from the electroceutical manufacturer, and when the electroceutical is out of order, request the after-sales service to the corresponding electroceutical manufacturer and send the repaired electroceutical to the corresponding user, thereby forming a continuous electroceutical distribution and supply chain of the electroceutical manufacturer and providing the user's convenience.

Additionally, according to an embodiment of the present disclosure, it is possible to satisfy the needs of the doctor, the electroceutical manufacturer and the user, by acting as connecting links among the doctor, the electroceutical manufacturer and the user with respect to the electroceutical pharmacy running online and offline.

The present disclosure has been hereinabove described with reference to the embodiments shown in the accompanying drawings, this is provided by way of illustration, and those having ordinary skill in the corresponding technical field will understand that a variety of other modifications and equivalents may be made thereto. Accordingly, the true technical protection scope of the present disclosure should be defined by the technical spirit of the appended claims.

[Detailed Description of Main Elements]

| | |
|---|---|
| 100: User terminal | 200: Electroceutical pharmacy server |
| 210: Input unit | 220: Stock status checking unit |
| 230: Stock status database | 240: Information providing and |
| | request unit |
| 250: Electroceutical providing unit | 260: User information database |
| 270: After-sales service unit | 300: Electroceutical manufacturer |
| | server |

What is claimed is:

1. A computer-implemented method for providing an online based electroceutical service by an electroceutical pharmacy server having a processor and a memory, the computer-implemented method comprising:
receiving an inquiry regarding an electroceutical and an input electroceutical prescription from a user terminal;
performing, by a preprocessing processor, natural language preprocessing (NLP) on the inquiry;
searching, by a query analysis processor, requested information from an external server;
providing, by an information search processor, the requested information in reply to the inquiry;
determining a stock status of the electroceutical included in the input electroceutical prescription;
transmitting a available supply date of the electroceutical to the user terminal according to the stock status, and requesting a pickup information input; and
providing the electroceutical according to the pickup information input when a purchase cost of the electroceutical is paid and pickup information including a pickup date and a pickup method is input from the user terminal.

2. The computer-implemented method of claim 1,
wherein the electroceutical is a medical device configured to apply microcurrent stimulation,
wherein the external server includes an online academic DB storing disease names, electroceutical names, guidelines, and efficacy information,
wherein the preprocessing processor performs the NLP on a text of the inquiry, in which the NLP includes morphological analysis, and roots extraction and analysis;
wherein the query analysis processor is configured to perform semantic analysis of the text, and includes a transformer-based named entity recognition model, which includes a neural network that learns context and meaning of a sentence by tracking relationships in sequential data and detects changes in meaning by analyzing the meaning and relationship of distant elements in the text using a self-attention technique, and
wherein an information search processor processes the reply in real time according to a preset information search algorithm; performs keyword matching in an external server based on the named entity recognized by the query analysis processor; and provides the reply to the inquiry based on the matched keyword in a natural dialogue format.

3. The computer-implemented method of claim 1,
wherein the determining the stock status comprises when the electroceutical is out of stock, requesting electroceutical supply and an expected supply date to an electroceutical manufacturer server of the electroceutical, and
wherein the requesting the pickup information input comprises providing the user terminal with the available supply date of the electroceutical in a calendar or a list view to request the pickup information input, and
wherein the providing the electroceutical comprises:
when the inputted pickup method is parcel delivery, booking a parcel delivery dispatch to a pre-stored address included in a user information database to be delivered by the pickup date; and
when the inputted pickup method is user visit and pickup, providing the user terminal with store information of an electroceutical pharmacy store including an address and a contact number of the electroceutical pharmacy store.

4. The computer-implemented method of claim 1, wherein the providing the electroceutical comprises providing usage guidelines of the electroceutical together with the electroceutical according to an input device description method from the user terminal.

5. The computer-implemented method of claim 1, further comprising:
receiving, by the electroceutical pharmacy server, an after-sales service request input of the electroceutical from the user terminal;
determining whether an after-sales service is possible or not by referring to a purchase date and electroceutical information of the electroceutical included in purchase history information of a user information database;
when the after-sales service is possible as a result of the determination, requesting the after-sales service of the electroceutical to an electroceutical manufacturer server of the electroceutical, and receiving after-sales service completion information including an expected after-sales service completion date from the electroceutical manufacturer server; and
providing the user terminal with the received after-sales service completion information, and requesting an after-sales service pickup information input to the user terminal.

6. The computer-implemented method for providing an electroceutical service of claim 1, wherein the determining the stock status comprises, when the electroceutical is out of stock, generating a recommendation list of electroceuticals currently available for supply in an order of similarity of performance and efficacy information to the electroceutical by analyzing the performance and efficacy information of the electroceutical and referring to a stock status database, and providing the generated recommendation list to the user terminal.

7. A computer-implemented pharmacy platform system comprising:
an electroceutical pharmacy server, having a processor and a memory, configured to:
receive an inquiry regarding an electroceutical and an input electroceutical prescription from a user terminal
perform, by a preprocessing processor, natural language preprocessing (NLP) on the inquiry;
search, by a query analysis processor, requested information from an external server;
provide, by an information search processor, the requested information in reply to the inquiry;
transmit an available supply date of the electroceutical to the user terminal according to a stock status of the electroceutical included in the prescription; and
request a pickup information input to the user terminal and provide the electroceutical according to the inputted pickup information; and
an electroceutical manufacturer server configured to provide electroceutical supply information to the electroceutical pharmacy server.

8. The computer-implemented pharmacy platform system of claim 7,
wherein the electroceutical is a medical device configured to apply microcurrent stimulation,
wherein the external server includes an online academic DB storing disease names, electroceutical names, guidelines, and efficacy information,
wherein the preprocessing processor performs the NLP on a text of the inquiry, in which the NLP includes morphological analysis, and roots extraction and analysis;
wherein the query analysis processor is configured to perform semantic analysis of the text, and includes a transformer-based named entity recognition model, which includes a neural network that learns context and meaning of a sentence by tracking relationships in sequential data and detects changes in meaning by analyzing the meaning and relationship of distant elements in the text using a self-attention technique, and
wherein an information search processor processes the reply in real time according to a preset information search algorithm; performs keyword matching in an external server based on the named entity recognized by the query analysis processor; and provides the reply to the inquiry based on the matched keyword in a natural dialogue format.

9. The computer-implemented pharmacy platform system of claim 7,
wherein an input unit configured to receive the inputted prescription from the user terminal;
a stock status checking unit configured to determine the stock status of the electroceutical included in the prescription by referring to a stock status database; an information providing and requesting unit configured to transmit the available supply date of the electroceutical to the user terminal according to the determined stock status, and request the pickup information input; an electroceutical providing unit configured to provide the electroceutical according to the inputted pickup information when pickup information including a pickup date and a pickup method is input from the user terminal and a purchase cost of the electroceutical is paid;
wherein the information providing and requesting unit is further configured to provide the user terminal with the available supply date of the electroceutical in a calendar or a list view to request the pickup information input, and
wherein the electroceutical providing unit is further configured to:
when the inputted pickup method is parcel delivery, book a parcel delivery dispatch to a pre-stored address included in a user information database to be delivered by the pickup date; and
when the inputted pickup method is user visit and pickup, provide the user terminal with store information of an electroceutical pharmacy store including an address and a contact number of the electroceutical pharmacy store.

10. The computer-implemented pharmacy platform system of claim 8, wherein the stock status checking unit is further configured to request electroceutical supply and an expected supply date to an electroceutical manufacturer server of the electroceutical when the electroceutical is out of stock, and
wherein the electroceutical pharmacy server is further configured to provide usage guidelines of the electroceutical together according to an input device description method from the user terminal.

11. The computer-implemented pharmacy platform system of claim 8, wherein the user terminal is further configured to input an after-sales service request of an electroceutical that a user has purchased and the electroceutical manufacturer server is further configured to respond to the after-sales service request, and
wherein the electroceutical pharmacy server further comprises an after-sales service unit configured to:
determine whether an after-sales service is possible or not by referring to a purchase date and electroceutical information of the electroceutical included in purchase history information of a user information database when the after-sales service request is received from the user terminal; and
when the after-sales service is possible, request the after-sales service of the electroceutical to the electroceutical manufacturer server of the electroceutical, and receive after-sales service completion information including an expected after-sales service completion date from the electroceutical manufacturer server; and
provide the after-sales service completion information to the user terminal, and request an after-sales service pickup information input to the user terminal.

12. The computer-implemented pharmacy platform system of claim 8, wherein when the electroceutical is out of stock, the stock status checking unit is further configured to generate a recommendation list of electroceuticals currently available for supply in an order of similarity of performance and efficacy information to the electroceutical by analyzing the performance and efficacy information of the electroceutical and referring to a stock status database, and provide the generated recommendation list to the user terminal.
